(19) United States
(12) Patent Application Publication
(10) Pub. No.: US 2014/0193297 A1
(43) Pub. Date: Jul. 10, 2014

(54) ODOR ELIMINATING COVERINGS

(71) Applicant: Extreme Hunting Solutions, LLC, Centerville, IA (US)

(72) Inventor: Randall Ferman, Randolph, NJ (US)

(73) Assignee: EXTREME HUNTING SOLUTIONS, LLC, Centerville, IA (US)

(21) Appl. No.: 14/202,813

(22) Filed: Mar. 10, 2014

Related U.S. Application Data
(63) Continuation of application No. 13/399,683, filed on Feb. 17, 2012.
(60) Provisional application No. 61/444,307, filed on Feb. 18, 2011.

(51) Int. Cl. A61L 9/014

(52) U.S. Cl. CPC A61L 9/014
USPC 422/5

(57) ABSTRACT

The present invention includes various methods of using a covering for substantially eliminating an odor of a shoe, boot or backpack where, with the covering placed onto the shoe, boot or backpack, the shoe, boot or backpack can be used. The present invention also includes various methods for concealing a hunter from potential prey by substantially eliminating an of an odor underlying object.

### CROSS-REFERENCE TO RELATED APPLICATIONS

This application is a continuation of U.S. application Ser. No. 13/399,683, filed on Feb. 17, 2012, which claims the benefit of the filing date of U.S. Provisional Patent Application No. 61/444,307, filed Feb. 18, 2011, the disclosures of which are hereby incorporated herein by reference.

### BACKGROUND OF THE INVENTION

The sport of hunting typically demands that a hunter conceal themselves from being discovered by potential prey. Thus, a hunter takes various steps in an attempt to conceal oneself from the animal being hunted. A classic example of such is camouflage, wherein the hunter wears clothing designed to blend in with the surroundings, the idea of camouflage being that the potential prey cannot distinguish the hunter from their environment.

Certain prey, particularly deer, for example, can also detect the hunter by smell. Substantially eliminating “human” odors is important in hunting in order to minimize the risk of the potential prey smelling the hunter and fleeing the area. As such, another example of camouflage used by hunters includes sprays or the like which, once applied, are designed to mask the scent of the hunter. However, such sprays must be re-applied often, sometimes even multiple times during a single use.

Disposable wrappings are also used by some hunters to minimize the odor of the hunters. However, such wrappings may only be used a single time, and many require the aforementioned spray to assist in masking the odor of the hunter. Additionally, such wrappings are not capable of being used on certain equipment often used by a hunter, such as shoes, boots, backpacks, and the like.

### BRIEF SUMMARY OF THE INVENTION

In a first embodiment, the present invention may include a covering for substantially eliminating an odor from an underlying object, the covering may include a material which may substantially cover the underlying object, and a composition incorporated with the material adapted to substantially eliminate the odor of the underlying object.

The covering may be, for example, a shoe cover, a backpack cover, or the like, wherein the underlying object is a shoe, such as a tennis shoe, a boot, or the like, or a backpack, respectively. The material may be any durable material and may include, for example, nylon, canvas, cotton, spandex, polyester, Gore-Tex®, silk, or the like. The material may be sufficiently durable such that the covering may withstand multiple or repeated uses.

The composition may be, for example, activated carbon. The composition may be applied to the surface of the material, impregnated within the material, applied as a layer within the material, or the like. The composition may reduce the odor of the underlying object, substantially eliminate the odor of the underlying object, or completely eliminate the odor of the underlying object.

The composition may also be rechargeable for multiple uses of the covering. In one example, the composition is rechargeable by placing the covering in a clothes dryer at a high temperature for a sufficient amount of time, for example, about 30 minutes.

The covering may be used in outdoor activities such as, for example, hunting. In hunting, the substantial elimination of human odors may be important so as to minimize the chances of potential prey from smelling the hunter and fleeing the area.

In another embodiment, the present invention may be a method of manufacturing a covering for substantially eliminating an odor from an underlying object intended to be covered, the method may include incorporating a composition into a material, the composition adapted to substantially eliminate the odor of the underlying object; and forming a material to a shape adapted to substantially cover the underlying object.

The material may be any durable material and may include, for example, nylon, canvas, cotton, spandex, polyester, Gore-Tex®, silk, or the like. The material may be sufficiently durable such that the covering may withstand multiple uses. Further, the material may be selected to be sufficiently durable for multiple uses in outdoor activities, such as hunting or the like.

The composition may be applied to the surface of the material, impregnated within the material, applied as a layer within the material, or the like. The composition may reduce the odor of the underlying object, substantially eliminate the odor of the underlying object, or completely eliminate the odor of the underlying object. The composition may also be rechargeable for multiple uses of the covering.

The material may be formed to substantially cover, for example, a shoe, a backpack or the like. The material may further be formed to substantially cover, for example, a tennis shoe, a boot, such as a hiking or hunting boot, or the like, and additionally, if desired, at least a portion of a leg.

In yet another embodiment, the present invention may include a method of using a covering for substantially eliminating an odor of an underlying object, the method may include selecting a covering adapted to sufficiently cover the underlying object; placing the covering onto the underlying object such that the underlying object may be substantially covered and the odors of the underlying object are substantially eliminated; and using the underlying object with the covering remaining on the underlying object.

The method may further include the step of removing the covering from the underlying object. The method may yet further include the step of recharging the covering, wherein recharging the covering may ready the covering for a subsequent use to substantially eliminate an odor of an underlying object.

The covering may include activated carbon adapted to substantially eliminate the odor of the underlying object. The recharging step may include placing the covering in a clothes dryer to ready the activated carbon for a subsequent use.

In yet another embodiment, the present invention may include a system for reducing an odor emanating from an underlying object, the system including the underlying object including the odor emanating therefrom; and a covering for covering at least a portion of the underlying object, the covering comprising a material and a composition, the composition incorporated with the material adapted to reduce the odor emanating from the underlying object.

### BRIEF DESCRIPTION OF THE DRAWINGS

The attached figures illustrate various embodiments of a covering for substantially covering an underlying object. In these illustrations, the underlying objects are a shoe, such as a hiking or hunting boot, and a backpack.

FIGS. 1 and 2 illustrate one embodiment of the present invention, sized for use on a shoe.

FIG. 3 illustrates an example of an underlying object, being a backpack.

FIGS. 4 and 5 illustrate another embodiment of the present invention, sized for use on the backpack of FIG. 3.

### DETAILED DESCRIPTION

Generally, the present invention includes a covering for substantially covering an underlying object and for substantially eliminating an odor. For example, the covering may substantially prevent the odor from emanating from the underlying object and into the surrounding environment. Alternatively, for example, the covering may substantially neutralize the odor emanating from the underlying object such that the odor does not affect the surrounding environment. While the below embodiments disclose the underlying object as a shoe or a backpack, the underlying object may be any other object a user desires to be substantially covered such as to substantially eliminate the odor of such object.

One use of the covering of the present invention may be for hunting, such that the covering substantially eliminates the odor of a piece of equipment (a shoe or backpack, for example). While this exemplary use will be used throughout this disclosure, the coverings of the present invention may be used for other activities or uses other than for hunting where it is desirable to minimize odors emanating from an underlying object.

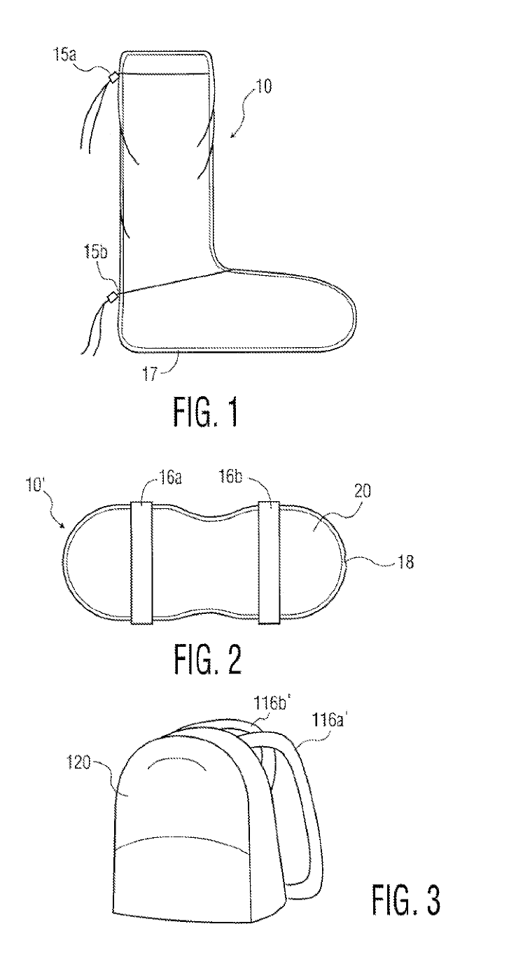

In a first embodiment, the present invention may include a cover for covering an underlying object to reduce an odor of the underlying object. The covering may reduce the odor of the underlying object by any desired amount along a range from, for example, partially reducing the odor of the underlying object, to substantially eliminating the odor of the underlying object. Moreover, the covering may cover a portion of the underlying object or may cover substantially the entire underlying object. In yet another arrangement, for example, as to a cover 10 for a shoe 20, illustrated in FIGS. 1 and 2, the cover 10 may substantially cover the shoe and a portion of a leg, such as at least a portion of a lower leg below the knee, extending from the shoe.

The covering 10 may be constructed of a material with sufficient durability to withstand multiple uses, for example, multiple days, weeks or even seasons of hunting. The material may be any durable material and may include, for example, nylon, canvas, cotton, spandex, polyester, Gore-Tex®, silk, or the like.

The material may have a color dependent upon the intended activity for which the covering will be used. Again, using the example of hunting, the material may have a color of, though not limited to, camouflage, brown, black, tan, blue, white, or any combination or pattern, on at least an outer layer of the covering. Of course, if the covering only includes one layer of material, than the entirety of the material may have such color.

The covering 10 may also have a composition incorporated in it which may be, for example, activated carbon (Scent-Lok Technologies, Muskegon, Mich.) or the like. The composition may be applied to the surface of the material, impregnated within the material, applied as a layer within the material, or the like. The composition may reduce the odor of the underlying object, substantially eliminate the odor of the underlying object, or completely eliminate the odor of the underlying object. Activated carbon, for example, may reduce the odor by neutralizing the odor and thus minimizing or eliminating the possibility of the odor escaping into the surrounding environment. The composition may also be rechargeable for multiple uses of the covering. In one example, the composition is rechargeable by placing the covering in a clothes dryer at a high temperature for a sufficient amount of time, for example, about 30 minutes.

In the embodiment of the covering 10, illustrated in FIG. 1, the covering 10 is for a shoe 20, such as a tennis shoe, hiking or hunting boot, or the like. The covering 10 may include material which substantially surrounds the boot 20 (extending above the ankle) and may further extend up a portion of a leg, such as at least the portion of the leg below a knee. The covering may include at least one cinching element 15a, 15b, such as an elastic cord, which may be tightened and secured to cinch the covering around the shoe, leg, or both. Other cinching elements such as Velcro® straps, cords or laces, button clasps, plastic bindings, or the like may also be used. An upper opening of the cover, through which the shoe, leg, foot, or any combination passes to be positioned within the cover, may be loose-fitting or may have an elastic band such that the opening may be expanded to allow passage of the shoe but may then constrict the opening to fit securely to the shoe, leg, or the like.

While the majority of the covering 10 may be constructed of the material as discussed above, the bottom 17 of the covering, covering the sole of the shoe 20, as in FIG. 1, may be rubber or have further cushion, support, or durable layers, to allow a user to walk, hike, stand, etc. while wearing the covering. Of course, the bottom of the covering may be constructed of the same material as the rest of the covering. At least a portion of the material of the covering, and preferably the majority of the material of the covering, may include a composition to reduce an odor emanating from the shoe, leg, or both. As above, the composition may be activated carbon.

Alternatively, a shoe covering 10′, illustrated in FIG. 2, may include straps 16a, 16b forming the bottom of the covering, rather than the bottom 17 illustrated in FIG. 1. In this embodiment, the covering may be open at the bottom such that no material is placed over the sole of the shoe. The shoe may, again, be any shoe such as but not limited to a tennis shoe, hiking or hunting boot, or the like. To assist in maintaining the placement of the covering on the shoe, the covering may include the at least one strap 16a, 16b which passes under the sole of the shoe to prevent the cover from migrating in an upward direction and off of the shoe. An open edge portion 18 of the covering of FIG. 2, similar to the upper end (discussed as to FIG. 1, above), may have an elastic band such that it fits around the perimeter of the shoe, similar to a shower cap. While this elastic band may lower the risk of the cover migrating in the upward direction, the at least one strap 16a, 16b will further reduce this risk. This type of strap may also be used as a cinching element elsewhere on the covering, or may further be used at the opening at the top of the covering by wrapping around the leg, shoe, or the like and holding the covering up on the leg, shoe, or the like. In this embodiment, even though the sole of the shoe is exposed, the rest of the shoe, and also the portion of the leg, is within the covering and thus the covering may reduce the odor of these underlying objects. If desired, the sole of the shoe may be treated with a separate spray, or the like, to reduce any odors emanating from the sole.

The various embodiments of the shoe covering may be sized to fit any shoe, such as a one-size-fits-all, or, alternatively, the covering may be sold in various sizes, e.g., small, medium, large, XL, and the like. Also, for example, the shoe coverings may be sized to fit shoes based on the intended activity, such as a covering sized for hiking shoes, hiking or hunting boots, tennis shoes, and the like.

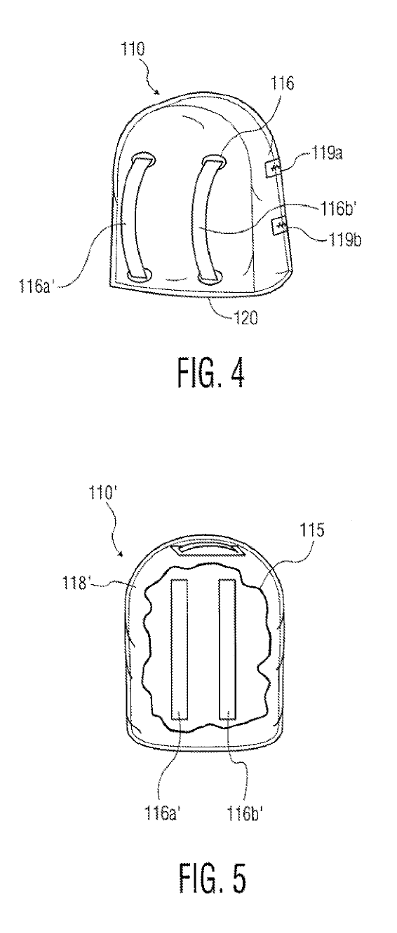

In another embodiment, as illustrated in FIGS. 3 and 4, the covering 110 may also be used to substantially cover a backpack 120. In this embodiment, the covering 110 may be sold in various sizes depending on the sizes of backpacks 120 such as, for example, light day-packs, overnight packs, trekking (multi-day) packs, gun packs, and the like. The covering may be constructed to substantially cover the backpack, but may, for example, include slits 116 to allow for the passage of a portion of the backpack, such as shoulder straps 116a′, 116b′, or the like, such that the backpack can still be used even with the covering in place. The slits may be closed and opened by a zipper, Velcro®, button clasps, or the like. The backpack may be placed within the covering through a closable opening in the covering such as, for example, through the bottom of the covering (not shown). The opening in the covering may be securely closed by a zipper, Velcro®, button clasps, or the like, such that the covering substantially covers the backpack, leaving, for example, only the shoulder straps exposed for ease of use with the covering in place. As with the shoe covering, above, the backpack covering may also include at least one cinching element to provide added securement and organization to the covering while placed on the backpack.

Furthermore, the covering may also include at least one access way 119a, 119b located on the covering 110 such that a user can gain access to at least one compartment of the backpack while leaving the covering in place over the backpack. An exemplary placement of the access way 119a, 119b is illustrated in FIG. 4, by which the opening is opened and closed via a zipper, Velcro®, button clasps, or other closure element may also be used. Of course, the access way, or more than one access way, may be of any size and in any location as desired. For example, if the covering were constructed with a particular backpack from a particular manufacturer in mind, then the access way or access ways of the covering may be sized and placed to optimize access to the compartment or compartments of the backpack with the covering in place.

In an alternate embodiment, where additional odor reduction is necessary, the covering itself may have shoulder straps. In this embodiment, the covering may not include slits to allow access to the shoulder straps of the backpack. This embodiment of the covering may further reduce odors which may, for example, emanate from the shoulder straps of the backpack by substantially sealing the entire backpack, including the shoulder straps, within the covering.

In yet a further backpack covering embodiment, illustrated in FIG. 5, the backpack covering 110′ may cover only a portion of the backpack, such that the covering is similar to a shower cap. In this embodiment, the top, bottom, sides and back of the backpack are substantially covered, but the front surface, including the shoulder straps, are not covered. The outer, open edge portion 118′ of the covering 110′ may include an elastic band 115 which may secure the covering to the backpack. Additionally, the covering may include straps, similar to the straps illustrated for the shoe covering in FIG. 2, which may wrap around the front of the backpack to provide further securement of the covering to the backpack.

Such coverings as discussed above may also be used for underlying objects other than shoes, legs and backpacks, and thus may be constructed to accommodate, for example, camping equipment, hunting equipment, other portions of the human body, the human body in total, or other objects which emanate an odor which needs to be reduced. While activated carbon is the disclosed composition for reducing the odors, other compositions may also be incorporated into the material of the covering which may have the same effect.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1.-19. (canceled)

20. A method of using a covering for substantially eliminating an odor of a shoe or boot, the method comprising:
selecting a covering adapted to cover the shoe or boot;
placing the covering onto the shoe or boot such that the shoe or boot is substantially covered and the odors of the shoe or boot are substantially eliminated; and
using the shoe or boot with the covering remaining on the shoe or boot.

21. The method of claim 20, wherein the step of using the shoe or boot includes wearing the shoe or boot on a leg of a user.

22. The method of claim 21, wherein the step of using the shoe or boot includes the covering placed over at least a portion of the leg of the user.

23. The method of claim 22, wherein the step of using the shoe or boot includes cinching a cinching element onto at least one of the shoe or boot and the portion of the leg.

24. The method of claim 20, wherein after the using step, further comprising the step of removing the covering from the shoe or boot.

25. The method of claim 24, wherein after the removing step, further comprising the step of recharging the covering, wherein recharging the covering prepares the covering for a subsequent use.

26. The method of claim 20, wherein the covering comprises activated carbon.

27. A method of concealing a hunter from potential prey by substantially eliminating an odor of an underlying object, the method comprising:
selecting a covering adapted to cover the underlying object;
placing the covering onto the underlying object to be used by the hunter such that the underlying object is substantially covered and the odors of the underlying object are substantially eliminated; and
using the underlying object with the covering remaining on the underlying object.

28. The method of claim 27, wherein the underlying object is a shoe, a boot or a backpack.

29. The method of claim 28, wherein the step of using the underlying object comprises wearing the shoe, boot or backpack.

30. The method of claim 27, wherein after the using step, further comprising the step of removing the covering from the underlying object.

31. The method of claim 30, wherein after the removing step, further comprising the step of recharging the covering, wherein recharging the covering may ready the covering for a subsequent use.

32. The method of claim 27, wherein the covering comprises activated carbon.

33. A method of using a covering for substantially eliminating an odor of a backpack, the method comprising:
selecting a covering adapted to cover the backpack;
placing the covering onto the backpack such that the backpack is substantially covered and the odors of the backpack are substantially eliminated; and
using the backpack with the covering remaining on the backpack.

34. The method of claim 33, wherein the step of placing the covering onto the backpack to substantially cover the backpack includes allowing access by a user to at least one strap of the backpack for the using step.

35. The method of claim 34, wherein the covering includes at least one access way or slit such that a user can access the backpack through the accessway and/or slit during use.

36. The method of claim 33, wherein after the using step, further comprising the step of removing the covering from the backpack.

37. The method of claim 36, wherein after the removing step, further comprising the step of recharging the covering, wherein recharging the covering may ready the covering for a subsequent use.

38. The method of claim 33, wherein the covering comprises activated carbon.
